SEQUENCE LISTING (47 sequences)

SEQ ID NO: 1 - DNA, 1521 bp, synthetic construct
  source 1..1521: mol_type = other DNA
atgaacaccattaatgaatacttgagtttagaagaattcgaagcaataatcttcggtaaccaaaaagtaactatctctgatgttgtcgtaaacagagttaacgaaagttttaacttcttaaaggaattttctggtaataaggttatatatggtgtaaacactggtttcggtccaatggctcaatacagaatcaaggaatctgatcaaatccaattgcaatacaatttgataagaagtcattcttcaggtactggtaaaccattatctcctgtttgtgctaaggctgcaatcttggcaagattgaacacattgtctttaggcaactcaggtgttcacccatctgttattaatttgatgtctgaattgataaacaaagacatcactcctttgatattcgaacatggtggtgttggtgcatctggtgacttggtccaattgtcccacttggccttagtattgataggtgaaggtgaagttttctataaaggtgaaagaagaccaacacctgaagtcttcgaaatcgaaggtttaaagcctatacaagtagaaatcagagaaggtttagctttgattaatggtacttctgtcatgacaggtataggtgttgtcaacgtataccatgctaagaaattgttggattggtcattgaagtccagttgtgccattaatgaattggttcaagcatatgatgaccatttctctgcagaattgaaccaaaccaagagacacaagggtcaacaagaaatcgcattgaagatgagacaaaatttgtccgatagtacattgatcagaaagagagaagaccacttatactcaggtgaaaacaccgaagaaattttcaaagaaaaggttcaagaatactactccttgagatgcgtcccacaaatcttgggtcctgtattggaaactattaataacgttgcctcaatcttggaagatgaattcaattccgctaacgataacccaatcatcgacgttaaaaatcaacatgtttatcacggtggtaacttccatggtgactacatttctttagaaatggacaaattgaagatagttatcacaaaattgaccatgttggctgaaagacaattgaactacttgttgaactcaaagattaacgaattgttgccacctttcgttaatttgggtacattgggttttaacttcggtatgcaaggtgttcaattcaccgccacttcaactacagctgaatcccaaatgttgagtaacccaatgtacgttcattccatccctaacaacaacgataaccaagacatcgtctctatgggtaccaactcagccgtcattacttccaaagtaatagaaaacgcattcgaagttttggccatcgaaatgatcacaattgtccaagctatcgattacttgggtcaaaaggacaagatctcttctgtttctaagaaatggtacgatgaaataagaaacataatcccaacctttaaggaagaccaagttatgtaccctttcgtacaaaaggttaaggatcatttgattaacaattaa

SEQ ID NO: 2 - DNA, 2093 bp, synthetic construct
  source 1..2093: mol_type = other DNA
atgaaaccagaagatttccgcgccagtacccaacgtcctttcaccggggaagagtatctgaaaagcctgcaggatggtcgcgagatctatatctatggcgagcgagtgaaagacgtcaccactcatccggcatttcgtaatgcggcagcgtctgttgcccagctgtacgacgcactgcacaaaccggagatgcaggactctctgtgttggaacaccgacaccggcagcggcggctatacccataaattcttccgcgtggcgaaaagtgccgacgacctgcgccagcaacgcgacgccatcgctgagtggtcacgcctgagctatggctggatgggccgtaccccagactacaaagccgctttcggttgcgcactgggcgcgaatccgggcttttacggtcagttcgagcagaacgcccgtaactggtacacccgtattcaggaaactggcctctactttaaccacgcgattgttaacccaccgatcgatcgtcatttgccgaccgataaagtgaaagacgtttacatcaagctggaaaaagagactgacgccgggattatcgtcagcggtgcgaaagtggttgccaccaactcggcgctgactcactacaacatgattggcttcggctcggcacaagtgatgggcgaaaacccggacttcgcactgatgttcgttgcgccaatggatgccgatggcgtgaaattaatctcccgcgcctcttatgagatggtcgcgggtgctaccggctcgccatacgactacccgctctccagccgcttcgatgagaacgatgcgattctggtgatggataacgtgctgattccatgggaaaacgtgctgatctaccgcgattttgatcgctgccgtcgctggacgatggaaggcggttttgcccgtatgtatccgctgcaagcctgtgtgcgcctggcagtgaaattagacttcattacggcactgctgaaaaaatcactcgaatgtaccggcaccctggagttccgtggtgtgcaggccgatctcggtgaagtggtagcgtggcgcaacaccttctgggcattgagtgactcgatgtgttcagaagcaacgccgtgggtcaacggggcttatttaccggatcatgccgcactgcaaacctatcgcgtactggcaccaatggcctacgcgaagatcaaaaacattatcgaacgcaacgttaccagtggcctgatctatctcccttccagtgcccgtgacctgaataatccgcagatcgaccagtatctggcgaagtatgtgcgcggttcgaacggtatggatcacgtccagcgcatcaagatcctcaaactgatgtgggatgctattggcagcgaatttggtggtcgtcacgaactgtatgaaatcaactactccggtagccaggatgagattcgcctgcagtgtctgcgccaggcacaaaactccggcaatatggacaagatgatggcgatggttgatcgctgcctgtcggaatacgaccaggacggctggactgtgccgcacctgcacaacaacgacgatatcaacatgctggataagctgctgaaataacgcagcaggaggttaagatgcaattagatgaacaacgcctgcgctttcgtgacgcgatggccagcctgtcggcagcggtaaatattatcaccaccgagggcgacgccggacaatgcgggattacggcaacggccgtctgctcggtcacggatacaccaccgtcgctgatggtgtgcattaacgccaacagtgcgatgaacccggtttttcagggcaacggcaagttgtgcgtcaacgtcctcaaccatgagcaggaactgatggcacgccacttcgcgggcatgacaggcatggcgatggaagagcgttttagcctctcatgctggcaaaaaggtccgctggcgcagccggtgctaaaaggttcgctggccagtcttgaaggtgagatccgcgatgtgcaggcaattggcacacatctggtgtatctggtggagattaaaaacatcatcctcagtgcagaaggtcatggacttatctactttaaacgccgtttccatccggtgatgctggaaatggaagctgcgatttaa

SEQ ID NO: 3 - DNA, 867 bp, synthetic construct
  source 1..867: mol_type = other DNA
atggatgttaccgcaaaatacgaattgattgggttgatggcttatcctatccgccacagtttatcgcctgaaatgcagaataaagccttagaaaaagcgggattgccatttacctatatggccttcgaagtggataacgatagctttcccggagcaattgaaggattaaaagctctaaaaatgcgcggaactggtgtatcaatgccgaacaaacaactggcgtgtgaatatgttgatgaattaacgccagcagccaaactggtgggtgccatcaacaccatcgttaatgatgatggctacctgcgcggctataacaccgacggcacgggccatattcgagccattaaagagagcggttttgatatcaaaggcaaaacaatggtgctgttaggggccggaggtgcctcaacggcgattggcgcgcagggggcaattgaaggtttaaaagaaattaaactctttaaccgtcgggatgagttcttcgataaagccctcgccttcgcgcagcgggttaacgaaaacaccgattgtgtcgtcacggttaccgatctcgccgatcagcaagcctttgctgaagccctggcttccgccgacattttaaccaatggcacaaaagtgggtatgaaaccccttgagaatgaatcattggttaatgatatcagtctgttacatccgggactgctggtcactgaatgcgtgtataacccgcatatgacgaagttattgcagcaggcgcaacaagctggttgcaaaacgattgatggatacggcatgttgttgtggcaaggggctgaacagttcacgttatggactggcaaagatttccctctggaatatgttaaacaggtcatggggttcggtgcctga

SEQ ID NO: 4 - DNA, 1311 bp, synthetic construct
  source 1..1311: mol_type = other DNA
atgggctctgaaaaaatgatgaaaatcaacattaaagaatctaccctggttaaaccgtctaaaccgaccccgaccaaacgtctgtggtcctctaacctggatctgatcgttggccgcattcacctgctgaccgtgtacttttataaaccgaacggtagctctaacttcttcgattctaaaatcatgaaagaagcgctgagcaacgttctggtgagcttctatccgatggctggccgtctggctcgtgatgaacagggtcgtattgaaattaactgcaacggcgaaggcgtgctgttcgttgaagctgaatctgatgctttcgttgatgatttcggcgatttcaccccgtccctggaactgcgcaaactgatcccgactgttgatacctccggcgatatctctacctttccgctgattatcttccaggtgacccgtttcaaatgtggtggtgtttctctgggcggcggcgtgttccacactctgtctgatggtctgagcagcatccacttcatcaacacctggtctgatatcgcgcgtggcctgagcgttgcaatcccgccgttcatcgatcgtaccctgctgcgcgcacgtgatccgccgacctcctccttcgaacacgttgaataccacccgccgccgagcctgatcagcagctctaaaagcctggaatctacgagcccgaaaccgagcaccaccaccatgctgaaattctcctctgaccagctgggtctgctgaaatctaaaagcaaacacgatggctctacctatgaaattctggcggcgcatatctggcgttgcacctgcaaagcacgtgccctgagcgatgatcagctgaccaaactgcacgttgcgactgacggccgtagccgcctgtgcccgccgctgccaccgggctacctgggtaacgttgtgttcaccggtaccccgatggctaaaagcagcgaactgctgcaggaaccgctgaccaacagcgcgaaacgcatccacagcgcgctgtccaaaatggacgataactacctgcgttccgcactggattacctggaactgctgccggatctgagcgcgctgatccgtggcccgacctacttcgcgagcccgaacctgaacatcaactcctggactcgtctgccggtgcacgattctgatttcggttggggccgtccgatccacatgggtccggcatgcatcctgtacgaaggtaccgtttacatcctgccaagcccgaacagcaaagaccgtaacctgcgtctggcagtttgtctggatgctgatcacatgccgctgtttgaaaaatacctgtacgaattctaa

SEQ ID NO: 5 - DNA, 1686 bp, synthetic construct
  source 1..1686: mol_type = other DNA
atggcgccgcaggaacaggcggtttctcaggttatggaaaaacagtctaacaacaacaacagcgacgttatcttccgtagcaaactgccggatatttacatcccgaaccatctgtctctgcacgattacatctttcagaacatttctgaatttgcgactaaaccgtgcctgattaacggcccgaccggtcacgtgtacacctacagcgatgttcacgtgatcagccgccagattgcggccaacttccacaaactgggcgtgaaccagaacgatgttgtgatgctgctgctgccgaactgcccggaatttgttctgtctttcctggcggcaagctttcgtggtgctaccgcaaccgcggctaacccgttcttcaccccggcggaaatcgctaaacaggcaaaagcttctaacactaaactgatcatcactgaagcgcgctatgttgataaaatcaaaccgctgcagaacgatgacggtgttgttattgtgtgcatcgatgataacgaatccgtgccaatcccggaaggctgcctgcgtttcaccgaactgacccagtccaccaccgaagcgtccgaagtgattgatagcgtggaaatttctccggatgatgtagtggcgctgccgtactctagcggtactaccggtctgccgaaaggtgttatgctgacccataaaggtctggttacttccgttgcacagcaggttgatggtgaaaacccgaacctgtacttccactctgatgatgtgatcctgtgcgttctgccgatgttccacatctacgcactgaactctattatgctgtgtggcctgcgtgtaggtgctgcaattttgatcatgccgaaattcgaaatcaacctgttgctggaactgatccagcgttgcaaagtgaccgttgcgccgatggttccgccgatcgttctggcaattgctaaaagctctgaaaccgaaaaatacgacctgagctctatccgcgttgttaaaagcggtgcggcaccgctgggtaaagaactggaagatgctgttaacgctaaattcccgaacgcgaaactgggccagggttacggcatgaccgaagctggtccggtgctggcgatgagcctgggcttcgctaaagaaccgtttccggttaaatccggcgcatgtggtaccgtggttcgtaacgctgaaatgaaaatcgttgatccggataccggtgatagcctgtctcgtaaccagccgggtgaaatttgcattcgtggtcatcagatcatgaaaggctatctgaacaatccggcggcgaccgcggaaactatcgataaagatggctggctgcacaccggtgacatcggtctgatcgatgacgatgatgaactgtttatcgtagaccgtctgaaagaactgattaaatacaaaggcttccaggtggcgccggctgaactggaagctctgctgatcggccacccggatatcaccgatgttgcggtggttgcgatgaaagaagaagcggcgggtgaagttccggttgcgttcgttgttaaatctaaagactctgaactgagcgaagatgatgttaaacagttcgtgagcaaacaggttgtgttttataaacgtattaacaaagttttcttcaccgaaagcatcccgaaagcgccgtctggcaaaatcctgcgtaaagacctgcgtgcgaaactggcgaacggcctgtaa

SEQ ID NO: 6 - DNA, 1053 bp, synthetic construct
  source 1..1053: mol_type = other DNA
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcaatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 7 - DNA, 882 bp, synthetic construct
  source 1..882: mol_type = other DNA
atgaccgttttcaaacacatcgcgatcatcggcctgggtctgatcggcagcagcgctgcgcgtgctaccaaagcgtactgcccggatgttaccgtgagcctgtacgataaaagcgaattcgtttgcgatcgtgcgcgtgcgctgaacctgggtgataacgtgaccgacgatatccaggatgcggttcgtgaagcggatctggttctgctgtgcgttccggttcgcgcgatgggtatcgttgcggcggcgatggcgccggcgctgaaaaaagatgttatcatctgcgataccggttctgttaaagtttctgttatcaaaaccctgcaggacaacctgccgaaccacatcattgttccgagccacccgctggcgggtaccgaaaacaacggtccggatgcaggcttcgcggaactgttccaggatcacccggttatcctgaccccggatgcgcacaccccggcgcaggcgatcgcgtacatcgcggattactgggaagaaatcggcggccgtattaacctgatgagcgcggaacaccacgatcacgttctggcgctgacctctcacctgccgcacgttatcgcgtaccagctgatcggcatggtttctggctacgaaaagaaatctcgtaccccgatcatgcgttacagcgcgggttctttccgtgatgcgacccgtgttgcggcgtctgaaccgcgtctgtggcaggacatcatgctggaaaacgcgccggctctgctgccggttctggatcacttcatcgctgatctgaaaaaactgcgtaccgcgatcgcgtcccaggatgaagattacctgctggaacacttcaaagaatctcagaaagcgcgtctggcgctgaaaaccgatcacgatatccacccgtaa

SEQ ID NO: 8 - DNA, 1089 bp, synthetic construct
  source 1..1089: mol_type = other DNA
atggagaggattgtcgttactctcggggaacgtagttacccaattaccatcgcatctggtttgtttaatgaaccagcttcattcttaccgctgaaatcgggcgagcaggtcatgttggtcaccaacgaaaccctggctcctctgtatctcgataaggtccgcggcgtacttgaacaggcgggtgttaacgtcgatagcgttatcctccctgacggcgagcagtataaaagcctggctgtactcgataccgtctttacggcgttgttacaaaagccgcatggtcgcgatactacgctggtggcgcttggcggcggcgtagtgggcgatctgaccggcttcgcggcggcgagttatcagcgcggtgttcgtttcattcaagtcccgacgacgttactgtcgcaggtcgattcctccgttggcggcaaaactgcggtcaaccatcccctcggtaaaaacatgattggcgcgttctaccagcctgcttcagtggtggtggatctcgactgtctgaaaacgcttcccccgcgtgagttagcgtcggggctggcagaagtcatcaaatacggcattattcttgacggtgcgttttttaactggctggaagagaatctggatgcgttgttgcgtctggacggtccggcaatggcgtactgtattcgccgttgttgtgaactgaaggcagaagttgtcgccgccgacgagcgcgaaaccgggttacgtgctttactgaatctgggacacacctttggtcatgccattgaagctgaaatggggtatggcaattggttacatggtgaagcggtcgctgcgggtatggtgatggcggcgcggacgtcggaacgtctcgggcagtttagttctgccgaaacgcagcgtattataaccctgctcaagcgggctgggttaccggtcaatgggccgcgcgaaatgtccgcgcaggcgtatttaccgcatatgctgcgtgacaagaaagtccttgcgggagagatacgcttaattcttccgttggcaattggtaagagtgaagttcgcagcggcgtttcgcacgagcttgttcttaacgccattgccgattgtcaatcagcgtaa

SEQ ID NO: 9 - DNA, 1104 bp, synthetic construct
  source 1..1104: mol_type = other DNA
atggaccgcattattcaatcaccgggtaaatacatccagggcgctgatgtgattaatcgtctgggcgaatacctgaagccgctggcagaacgctggttagtggtgggtgacaaatttgttttaggttttgctcaatccactgtcgagaaaagctttaaagatgctggactggtagtagaaattgcgccgtttggcggtgaatgttcgcaaaatgagatcgaccgtctgcgtggcatcgcggagactgcgcagtgtggcgcaattctcggtatcggtggcggaaaaaccctcgatactgccaaagcactggcacatttcatgggtgttccggtagcgatcgcaccgactatcgcctctaccgatgcaccgtgcagcgcattgtctgttatctacaccgatgagggtgagtttgaccgctatctgctgttgccaaataacccgaatatggtcattgtcgacaccaaaatcgtcgctggcgcacctgcacgtctgttagcggcgggtatcggcgatgcgctggcaacctggtttgaagcgcgtgcctgctctcgtagcggcgcgaccaccatggcgggcggcaagtgcacccaggctgcgctggcactggctgaactgtgctacaacaccctgctggaagaaggcgaaaaagcgatgcttgctgccgaacagcatgtagtgactccggcgctggagcgcgtgattgaagcgaacacctatttgagcggtgttggttttgaaagtggtggtctggctgcggcgcacgcagtgcataacggcctgaccgctatcccggacgcgcatcactattatcacggtgaaaaagtggcattcggtacgctgacgcagctggttctggaaaacgcgccggtggaggaaatcgaaaccgtagctgcgcttagccatgcggtaggtttgccaataactctcgctcaactggatattaaagaagatgtcccggcgaaaatgcgaattgtggcagaagcggcatgtgcagaaggtgaaaccatccacaacatgcctggcggcgcgacgccagatcaggtttacgccgctctgctggtagccgaccagtacggtcagcgtttcctgcaagagtgggaataa

SEQ ID NO: 10 - DNA, 1308 bp, synthetic construct
  source 1..1308: mol_type = other DNA
atgaacattaccatcacgaaatcctctctgatcccgccgtctgaaaccctgccgggtagcgaaacccgcctgtggacttctaacctggatttagtagtaggtcgtatccacattctgaccgtttacttctatcgcccgaacggttcttctaacttctttgatccgaacgcgatgaaacaggcgctggcggatgttctggttagcttctatccgatggcgggtcgtctgggtcgtgatgaatctggccgcgttgttatcaactgtaacggcgaaggtgcgctgttcgttgaagctgaagcggatagctgtctggatgatttcggcgaattcaccccgagcccggaactgcgttctctgactccggcggttgattacagcggcgatgtttccagctatccgctgttcttcgcgcaggttacccacttcaaatgcggcggtgtttccctgggttgcggcgtgttccacactctgagcgatggtctgagcagcctgcacttcattaacacctggtctgatgttgcgcgtggtctgagcgtagcgatcccgccgttcattgaccgtaccctgctgcgtgcgcgtgaaccgccgaccccgacccatgaccacgttgaataccacccgccgccgaccatgaccatgctgaacaccatccagaaaccgggcccgctgtccaaatcgtccaccaccatgctgaaactgaccctggaccagctgaacaccctgaaagcaaaaaccaaaaccgaaggtggcccgagctacagcacctacgaaatcctggctgcacacatctggcgctgtgcgtgcaaagcgcgcggcctgccggatgatcagctgaccaaactgtacgtggcgaccgacggtcgtagccgcctgagcccgcatctgccgcgtggctatctgggtaacgtcgttttcacagcgaccccggttgcaacttccggcgctctgacctcggactctctgcagaacgcggctaaactgattcacaccaccctgacccagatggataacgaatacctgcgctctgcgatcgactacctggaaatccagccggatctgagcgccctgatccgtggtccgtcgtactttgcgtccccgaacctgaacatcaacgcgtggtcacgtctgccggttcacgatgcggattttggttggggtcgtccggtttttatgggtccggctatcatcctgtatgaaggtaccgtttacgtactgccgagcccgaacaacgatcgtagcgttagcctggcggtttgcctggatgctaaagaacagccgctgtttgaaaaatatctgtatgaattctaa

SEQ ID NO: 11 - DNA, 1332 bp, synthetic construct
  source 1..1332: mol_type = other DNA
atgggctctgatcagaaaatgatgatgaacatcgatatcatgaaatctagcatcgttccgccgagcgaactgattgcggattgcccgaaacagctgtggacctctaacctggatctggttgttggtcgtatccacatcctgaccgtttacttctaccgtccgaacggctctagcaaattcttcgatccgaacgttatgaaaaaagcgctggctgatgttctggtgagcttctacccgatggcgggccgtctgggtcgtgatgaaaccgatcgtatcgttatcaactgcaacaacgaaggcgttctgttcgttgaagctgaaagcgacagcaccctggatgacttcggcgaactgaaaccgagcccggaattccgtcagctgaccccgagcgttgattactctggcgacattagctcctacccgctgttcttcgcgcaggtgacccactttaaatgtggtggcgttgcgctgggttgcggcgttcaccacaccctgagcgatggcctgtctagcctgcacttcatcaacacctggagcgatatggcgcgtggcctgagcgtggcgatcccgccgttcatcgaacgcaccctgctgcgtgcacgtgaaccgccgaccccgacctacgatcacgttgaataccacagcccgccgagcatgaacaccaccgcgcagaaaccgggcagcggtagcctgagcaaaagcagcaccaccatgctgaaactgaccctggatcagctgaacagcctgaaagctaaagcgaaaagcgaaagcggttctacccactccacctacgaaatcctggcggcgcacatctggcgttgcgcgtgcaaagcgcgtggcctgccggatgatcagctgagcaaactgtacgttgcgaccgatggccgttctcgtctgtctccgcgtctgccgccgggctacctgggtaacgttgttttcaccgcgaccccggtggcaaaaagcggtgacctgaccagcaaaagcctgagcaacaccgcgaaactgatccacactaccctgaccaaaatggatgatgattacctgcgttctgcgatcgactatctggaatctcagccggatctgtctgcgctgatccgtggtccgtcttacttcgcgagtccgaacctgaacattaacgcttggactcgtctgccggtttatgatgcagatttcggttggggccgtccgatcttcatgggcccggcatgcatcctgtatgaaggtaccatttatgttctgcctagccctaacaatgatcgtagcgtttccttagcagtttgtctggatgccaacgagcagccgctgtttgaaaaattcctgtacgaattctaa

SEQ ID NO: 12 - protein, 506 aa, Flavobacterium johnsoniae
MNTINEYLSLEEFEAIIFGNQKVTISDVVVNRVNESFNFLKEFSGNKVIYGVNTGFGPMAQYRIKESDQIQLQYNLIRSHSSGTGKPLSPVCAKAAILARLNTLSLGNSGVHPSVINLMSELINKDITPLIFEHGGVGASGDLVQLSHLALVLIGEGEVFYKGERRPTPEVFEIEGLKPIQVEIREGLALINGTSVMTGIGVVNVYHAKKLLDWSLKSSCAINELVQAYDDHFSAELNQTKRHKGQQEIALKMRQNLSDSTLIRKREDHLYSGENTEEIFKEKVQEYYSLRCVPQILGPVLETINNVASILEDEFNSANDNPIIDVKNQHVYHGGNFHGDYISLEMDKLKIVITKLTMLAERQLNYLLNSKINELLPPFVNLGTLGFNFGMQGVQFTATSTTAESQMLSNPMYVHSIPNNNDNQDIVSMGTNSAVITSKVIENAFEVLAIEMITIVQAIDYLGQKDKISSVSKKWYDEIRNIIPTFKEDQVMYPFVQKVKDHLINN

SEQ ID NO: 13 - protein, 520 aa, Escherichia coli
MKPEDFRASTQRPFTGEEYLKSLQDGREIYIYGERVKDVTTHPAFRNAAASVAQLYDALHKPEMQDSLCWNTDTGSGGYTHKFFRVAKSADDLRQQRDAIAEWSRLSYGWMGRTPDYKAAFGCALGANPGFYGQFEQNARNWYTRIQETGLYFNHAIVNPPIDRHLPTDKVKDVYIKLEKETDAGIIVSGAKVVATNSALTHYNMIGFGSAQVMGENPDFALMFVAPMDADGVKLISRASYEMVAGATGSPYDYPLSSRFDENDAILVMDNVLIPWENVLIYRDFDRCRRWTMEGGFARMYPLQACVRLAVKLDFITALLKKSLECTGTLEFRGVQADLGEVVAWRNTFWALSDSMCSEATPWVNGAYLPDHAALQTYRVLAPMAYAKIKNIIERNVTSGLIYLPSSARDLNNPQIDQYLAKYVRGSNGMDHVQRIKILKLMWDAIGSEFGGRHELYEINYSGSQDEIRLQCLRQAQNSGNMDKMMAMVDRCLSEYDQDGWTVPHLHNNDDINMLDKLLK

SEQ ID NO: 14 - protein, 170 aa, Escherichia coli
MQLDEQRLRFRDAMASLSAAVNIITTEGDAGQCGITATAVCSVTDTPPSLMVCINANSAMNPVFQGNGKLCVNVLNHEQELMARHFAGMTGMAMEERFSLSCWQKGPLAQPVLKGSLASLEGEIRDVQAIGTHLVYLVEIKNIILSAEGHGLIYFKRRFHPVMLEMEAAI

SEQ ID NO: 15 - protein, 288 aa, Escherichia coli
MDVTAKYELIGLMAYPIRHSLSPEMQNKALEKAGLPFTYMAFEVDNDSFPGAIEGLKALKMRGTGVSMPNKQLACEYVDELTPAAKLVGAINTIVNDDGYLRGYNTDGTGHIRAIKESGFDIKGKTMVLLGAGGASTAIGAQGAIEGLKEIKLFNRRDEFFDKALAFAQRVNENTDCVVTVTDLADQQAFAEALASADILTNGTKVGMKPLENESLVNDISLLHPGLLVTECVYNPHMTKLLQQAQQAGCKTIDGYGMLLWQGAEQFTLWTGKDFPLEYVKQVMGFGA

SEQ ID NO: 16 - protein, 436 aa, Nicotiana tabacum
MGSEKMMKINIKESTLVKPSKPTPTKRLWSSNLDLIVGRIHLLTVYFYKPNGSSNFFDSKIMKEALSNVLVSFYPMAGRLARDEQGRIEINCNGEGVLFVEAESDAFVDDFGDFTPSLELRKLIPTVDTSGDISTFPLIIFQVTRFKCGGVSLGGGVFHTLSDGLSSIHFINTWSDIARGLSVAIPPFIDRTLLRARDPPTSSFEHVEYHPPPSLISSSKSLESTSPKPSTTTMLKFSSDQLGLLKSKSKHDGSTYEILAAHIWRCTCKARALSDDQLTKLHVATDGRSRLCPPLPPGYLGNVVFTGTPMAKSSELLQEPLTNSAKRIHSALSKMDDNYLRSALDYLELLPDLSALIRGPTYFASPNLNINSWTRLPVHDSDFGWGRPIHMGPACILYEGTVYILPSPNSKDRNLRLAVCLDADHMPLFEKYLYEF

SEQ ID NO: 17 - protein, 561 aa, Arabidopsis thaliana
MAPQEQAVSQVMEKQSNNNNSDVIFRSKLPDIYIPNHLSLHDYIFQNISEFATKPCLINGPTGHVYTYSDVHVISRQIAANFHKLGVNQNDVVMLLLPNCPEFVLSFLAASFRGATATAANPFFTPAEIAKQAKASNTKLIITEARYVDKIKPLQNDDGVVIVCIDDNESVPIPEGCLRFTELTQSTTEASEVIDSVEISPDDVVALPYSSGTTGLPKGVMLTHKGLVTSVAQQVDGENPNLYFHSDDVILCVLPMFHIYALNSIMLCGLRVGAAILIMPKFEINLLLELIQRCKVTVAPMVPPIVLAIAKSSETEKYDLSSIRVVKSGAAPLGKELEDAVNAKFPNAKLGQGYGMTEAGPVLAMSLGFAKEPFPVKSGACGTVVRNAEMKIVDPDTGDSLSRNQPGEICIRGHQIMKGYLNNPAATAETIDKDGWLHTGDIGLIDDDDELFIVDRLKELIKYKGFQVAPAELEALLIGHPDITDVAVVAMKEEAAGEVPVAFVVKSKDSELSEDDVKQFVSKQVVFYKRINKVFFTESIPKAPSGKILRKDLRAKLANGL

SEQ ID NO: 18 - protein, 350 aa, synthetic construct
MNYQNDDLRIKEIKELLPPVALLEKFPATENAANTVAHARKAIHKILKGNDDRLLVVIGPCSIHDPVAAKEYATRLLALREELKDELEIVMRVYFEKPRTTVGWKGLINDPHMDNSFQINDGLRIARKLLLDINDSGLPAAGEFLNMITPQYLADLMSWGAIGARTTESQVHRELASGLSCPVGFKNGTDGTIKVAIDAINAAGAPHCFLSVTKWGHSAIVNTSGNGDCHIILRGGKEPNYSAKHVAEVKEGLNKAGLPAQVMIDFSHANSSKQFKKQMDVCADVCQQIAGGEKAIIGVMVESHLVEGNQSLESGEPLAYGKSITDACIGWEDTDALLRQLANAVKARRG

SEQ ID NO: 19 - protein, 293 aa, Zymomonas mobilis
MTVFKHIAIIGLGLIGSSAARATKAYCPDVTVSLYDKSEFVCDRARALNLGDNVTDDIQDAVREADLVLLCVPVRAMGIVAAAMAPALKKDVIICDTGSVKVSVIKTLQDNLPNHIIVPSHPLAGTENNGPDAGFAELFQDHPVILTPDAHTPAQAIAYIADYWEEIGGRINLMSAEHHDHVLALTSHLPHVIAYQLIGMVSGYEKKSRTPIMRYSAGSFRDATRVAASEPRLWQDIMLENAPALLPVLDHFIADLKKLRTAIASQDEDYLLEHFKESQKARLALKTDHDIHP

SEQ ID NO: 20 - protein, 362 aa, Escherichia coli
MERIVVTLGERSYPITIASGLFNEPASFLPLKSGEQVMLVTNETLAPLYLDKVRGVLEQAGVNVDSVILPDGEQYKSLAVLDTVFTALLQKPHGRDTTLVALGGGVVGDLTGFAAASYQRGVRFIQVPTTLLSQVDSSVGGKTAVNHPLGKNMIGAFYQPASVVVDLDCLKTLPPRELASGLAEVIKYGIILDGAFFNWLEENLDALLRLDGPAMAYCIRRCCELKAEVVAADERETGLRALLNLGHTFGHAIEAEMGYGNWLHGEAVAAGMVMAARTSERLGQFSSAETQRIITLLKRAGLPVNGPREMSAQAYLPHMLRDKKVLAGEIRLILPLAIGKSEVRSGVSHELVLNAIADCQSA

SEQ ID NO: 21 - protein, 367 aa, Escherichia coli
MDRIIQSPGKYIQGADVINRLGEYLKPLAERWLVVGDKFVLGFAQSTVEKSFKDAGLVVEIAPFGGECSQNEIDRLRGIAETAQCGAILGIGGGKTLDTAKALAHFMGVPVAIAPTIASTDAPCSALSVIYTDEGEFDRYLLLPNNPNMVIVDTKIVAGAPARLLAAGIGDALATWFEARACSRSGATTMAGGKCTQAALALAELCYNTLLEEGEKAMLAAEQHVVTPALERVIEANTYLSGVGFESGGLAAAHAVHNGLTAIPDAHHYYHGEKVAFGTLTQLVLENAPVEEIETVAALSHAVGLPITLAQLDIKEDVPAKMRIVAEAACAEGETIHNMPGGATPDQVYAALLVADQYGQRFLQEWE

SEQ ID NO: 22 - DNA, 9109 bp, synthetic construct
  source 1..9109: mol_type = other DNA
ggggaattgtgagcggataacaattcccctgtagaaataattttgtttaactttaataaggagatataccatgggcatgaacaccattaatgaatacttgagtttagaagaattcgaagcaataatcttcggtaaccaaaaagtaactatctctgatgttgtcgtaaacagagttaacgaaagttttaacttcttaaaggaattttctggtaataaggttatatatggtgtaaacactggtttcggtccaatggctcaatacagaatcaaggaatctgatcaaatccaattgcaatacaatttgataagaagtcattcttcaggtactggtaaaccattatctcctgtttgtgctaaggctgcaatcttggcaagattgaacacattgtctttaggcaactcaggtgttcacccatctgttattaatttgatgtctgaattgataaacaaagacatcactcctttgatattcgaacatggtggtgttggtgcatctggtgacttggtccaattgtcccacttggccttagtattgataggtgaaggtgaagttttctataaaggtgaaagaagaccaacacctgaagtcttcgaaatcgaaggtttaaagcctatacaagtagaaatcagagaaggtttagctttgattaatggtacttctgtcatgacaggtataggtgttgtcaacgtataccatgctaagaaattgttggattggtcattgaagtccagttgtgccattaatgaattggttcaagcatatgatgaccatttctctgcagaattgaaccaaaccaagagacacaagggtcaacaagaaatcgcattgaagatgagacaaaatttgtccgatagtacattgatcagaaagagagaagaccacttatactcaggtgaaaacaccgaagaaattttcaaagaaaaggttcaagaatactactccttgagatgcgtcccacaaatcttgggtcctgtattggaaactattaataacgttgcctcaatcttggaagatgaattcaattccgctaacgataacccaatcatcgacgttaaaaatcaacatgtttatcacggtggtaacttccatggtgactacatttctttagaaatggacaaattgaagatagttatcacaaaattgaccatgttggctgaaagacaattgaactacttgttgaactcaaagattaacgaattgttgccacctttcgttaatttgggtacattgggttttaacttcggtatgcaaggtgttcaattcaccgccacttcaactacagctgaatcccaaatgttgagtaacccaatgtacgttcattccatccctaacaacaacgataaccaagacatcgtctctatgggtaccaactcagccgtcattacttccaaagtaatagaaaacgcattcgaagttttggccatcgaaatgatcacaattgtccaagctatcgattacttgggtcaaaaggacaagatctcttctgtttctaagaaatggtacgatgaaataagaaacataatcccaacctttaaggaagaccaagttatgtaccctttcgtacaaaaggttaaggatcatttgattaacaattaaaaggagatataccatgaaaccagaagatttccgcgccagtacccaacgtcctttcaccggggaagagtatctgaaaagcctgcaggatggtcgcgagatctatatctatggcgagcgagtgaaagacgtcaccactcatccggcatttcgtaatgcggcagcgtctgttgcccagctgtacgacgcactgcacaaaccggagatgcaggactctctgtgttggaacaccgacaccggcagcggcggctatacccataaattcttccgcgtggcgaaaagtgccgacgacctgcgccagcaacgcgacgccatcgctgagtggtcacgcctgagctatggctggatgggccgtaccccagactacaaagccgctttcggttgcgcactgggcgcgaatccgggcttttacggtcagttcgagcagaacgcccgtaactggtacacccgtattcaggaaactggcctctactttaaccacgcgattgttaacccaccgatcgatcgtcatttgccgaccgataaagtgaaagacgtttacatcaagctggaaaaagagactgacgccgggattatcgtcagcggtgcgaaagtggttgccaccaactcggcgctgactcactacaacatgattggcttcggctcggcacaagtgatgggcgaaaacccggacttcgcactgatgttcgttgcgccaatggatgccgatggcgtgaaattaatctcccgcgcctcttatgagatggtcgcgggtgctaccggctcgccatacgactacccgctctccagccgcttcgatgagaacgatgcgattctggtgatggataacgtgctgattccatgggaaaacgtgctgatctaccgcgattttgatcgctgccgtcgctggacgatggaaggcggttttgcccgtatgtatccgctgcaagcctgtgtgcgcctggcagtgaaattagacttcattacggcactgctgaaaaaatcactcgaatgtaccggcaccctggagttccgtggtgtgcaggccgatctcggtgaagtggtagcgtggcgcaacaccttctgggcattgagtgactcgatgtgttcagaagcaacgccgtgggtcaacggggcttatttaccggatcatgccgcactgcaaacctatcgcgtactggcaccaatggcctacgcgaagatcaaaaacattatcgaacgcaacgttaccagtggcctgatctatctcccttccagtgcccgtgacctgaataatccgcagatcgaccagtatctggcgaagtatgtgcgcggttcgaacggtatggatcacgtccagcgcatcaagatcctcaaactgatgtgggatgctattggcagcgaatttggtggtcgtcacgaactgtatgaaatcaactactccggtagccaggatgagattcgcctgcagtgtctgcgccaggcacaaaactccggcaatatggacaagatgatggcgatggttgatcgctgcctgtcggaatacgaccaggacggctggactgtgccgcacctgcacaacaacgacgatatcaacatgctggataagctgctgaaataaatgcaattagatgaacaacgcctgcgctttcgtgacgcgatggccagcctgtcggcagcggtaaatattatcaccaccgagggcgacgccggacaatgcgggattacggcaacggccgtctgctcggtcacggatacaccaccgtcgctgatggtgtgcattaacgccaacagtgcgatgaacccggtttttcagggcaacggcaagttgtgcgtcaacgtcctcaaccatgagcaggaactgatggcacgccacttcgcgggcatgacaggcatggcgatggaagagcgttttagcctctcatgctggcaaaaaggtccgctggcgcagccggtgctaaaaggttcgctggccagtcttgaaggtgagatccgcgatgtgcaggcaattggcacacatctggtgtatctggtggagattaaaaacatcatcctcagtgcagaaggtcatggacttatctactttaaacgccgtttccatccggtgatgctggaaatggaagctgcgatttaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcgccgcaggaacaggcggtttctcaggttatggaaaaacagtctaacaacaacaacagcgacgttatcttccgtagcaaactgccggatatttacatcccgaaccatctgtctctgcacgattacatctttcagaacatttctgaatttgcgactaaaccgtgcctgattaacggcccgaccggtcacgtgtacacctacagcgatgttcacgtgatcagccgccagattgcggccaacttccacaaactgggcgtgaaccagaacgatgttgtgatgctgctgctgccgaactgcccggaatttgttctgtctttcctggcggcaagctttcgtggtgctaccgcaaccgcggctaacccgttcttcaccccggcggaaatcgctaaacaggcaaaagcttctaacactaaactgatcatcactgaagcgcgctatgttgataaaatcaaaccgctgcagaacgatgacggtgttgttattgtgtgcatcgatgataacgaatccgtgccaatcccggaaggctgcctgcgtttcaccgaactgacccagtccaccaccgaagcgtccgaagtgattgatagcgtggaaatttctccggatgatgtagtggcgctgccgtactctagcggtactaccggtctgccgaaaggtgttatgctgacccataaaggtctggttacttccgttgcacagcaggttgatggtgaaaacccgaacctgtacttccactctgatgatgtgatcctgtgcgttctgccgatgttccacatctacgcactgaactctattatgctgtgtggcctgcgtgtaggtgctgcaattttgatcatgccgaaattcgaaatcaacctgttgctggaactgatccagcgttgcaaagtgaccgttgcgccgatggttccgccgatcgttctggcaattgctaaaagctctgaaaccgaaaaatacgacctgagctctatccgcgttgttaaaagcggtgcggcaccgctgggtaaagaactggaagatgctgttaacgctaaattcccgaacgcgaaactgggccagggttacggcatgaccgaagctggtccggtgctggcgatgagcctgggcttcgctaaagaaccgtttccggttaaatccggcgcatgtggtaccgtggttcgtaacgctgaaatgaaaatcgttgatccggataccggtgatagcctgtctcgtaaccagccgggtgaaatttgcattcgtggtcatcagatcatgaaaggctatctgaacaatccggcggcgaccgcggaaactatcgataaagatggctggctgcacaccggtgacatcggtctgatcgatgacgatgatgaactgtttatcgtagaccgtctgaaagaactgattaaatacaaaggcttccaggtggcgccggctgaactggaagctctgctgatcggccacccggatatcaccgatgttgcggtggttgcgatgaaagaagaagcggcgggtgaagttccggttgcgttcgttgttaaatctaaagactctgaactgagcgaagatgatgttaaacagttcgtgagcaaacaggttgtgttttataaacgtattaacaaagttttcttcaccgaaagcatcccgaaagcgccgtctggcaaaatcctgcgtaaagacctgcgtgcgaaactggcgaacggcctgtaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 23 - DNA, 7176 bp, synthetic construct
  source 1..7176: mol_type = other DNA
ggggaattgtgagcggataacaattcccctctagaaataattttgtttaactttaagaaggagatataccatgggcatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaaaaggagatataccatgaccgttttcaaacacatcgcgatcatcggcctgggtctgatcggcagcagcgctgcgcgtgctaccaaagcgtactgcccggatgttaccgtgagcctgtacgataaaagcgaattcgtttgcgatcgtgcgcgtgcgctgaacctgggtgataacgtgaccgacgatatccaggatgcggttcgtgaagcggatctggttctgctgtgcgttccggttcgcgcgatgggtatcgttgcggcggcgatggcgccggcgctgaaaaaagatgttatcatctgcgataccggttctgttaaagtttctgttatcaaaaccctgcaggacaacctgccgaaccacatcattgttccgagccacccgctggcgggtaccgaaaacaacggtccggatgcaggcttcgcggaactgttccaggatcacccggttatcctgaccccggatgcgcacaccccggcgcaggcgatcgcgtacatcgcggattactgggaagaaatcggcggccgtattaacctgatgagcgcggaacaccacgatcacgttctggcgctgacctctcacctgccgcacgttatcgcgtaccagctgatcggcatggtttctggctacgaaaagaaatctcgtaccccgatcatgcgttacagcgcgggttctttccgtgatgcgacccgtgttgcggcgtctgaaccgcgtctgtggcaggacatcatgctggaaaacgcgccggctctgctgccggttctggatcacttcatcgctgatctgaaaaaactgcgtaccgcgatcgcgtcccaggatgaagattacctgctggaacacttcaaagaatctcagaaagcgcgtctggcgctgaaaaccgatcacgatatccacccgtaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgttaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 24 - DNA, 52 bp, synthetic construct
  source 1..52: mol_type = other DNA
aagtataagaaggagatatacatatggatgttaccgcaaaatacgaattgat

SEQ ID NO: 25 - DNA, 25 bp, synthetic construct
  source 1..25: mol_type = other DNA
tcctttcaggcaccgaaccccatga

SEQ ID NO: 26 - DNA, 59 bp, synthetic construct
  source 1..59: mol_type = other DNA
ggttcggtgcctgaaaggagatataccatgggctctgaaaaaatgatgaaaatcaacat

SEQ ID NO: 27 - DNA, 53 bp, synthetic construct
  source 1..53: mol_type = other DNA
gtggcagcagcctaggttaattagaattcgtacaggtatttttcaaacagcgg

SEQ ID NO: 28 - DNA, 20 bp, synthetic construct
  source 1..20: mol_type = other DNA
ttaacctaggctgctgccac

SEQ ID NO: 29 - DNA, 45 bp, synthetic construct
  source 1..45: mol_type = other DNA
catatgtatatctccttcttatacttaactaatatactaagatgg

SEQ ID NO: 30 - DNA, 56 bp, synthetic construct
  source 1..56: mol_type = other DNA
gttcggtgcctgaaaggagatataccatgaacattaccatcacgaaatcctctctg

SEQ ID NO: 31 - DNA, 54 bp, synthetic construct
  source 1..54: mol_type = other DNA
gtggcagcagcctaggttaattagaattcatacagatatttttcaaacagcggc

SEQ ID NO: 32 - DNA, 46 bp, synthetic construct
  source 1..46: mol_type = other DNA
taaggagatataccatgggcatggagaggattgtcgttactctcgg

SEQ ID NO: 33 - DNA, 45 bp, synthetic construct
  source 1..45: mol_type = other DNA
cattatgcggccgcaagcttttacgctgattgacaatcggcaatg

SEQ ID NO: 34 - DNA, 22 bp, synthetic construct
  source 1..22: mol_type = other DNA
aagcttgcggccgcataatgct

SEQ ID NO: 35 - DNA, 32 bp, synthetic construct
  source 1..32: mol_type = other DNA
gcccatggtatatctccttattaaagttaaac

SEQ ID NO: 36 - DNA, 46 bp, synthetic construct; identical to SEQ ID NO: 32
  source 1..46: mol_type = other DNA

SEQ ID NO: 37 - DNA, 31 bp, synthetic construct
  source 1..31: mol_type = other DNA
ctccttttacgctgattgacaatcggcaatg

SEQ ID NO: 38 - DNA, 57 bp, synthetic construct
  source 1..57: mol_type = other DNA
ccgattgtcaatcagcgtaaaaggagatataccatggaccgcattattcaatcaccg

SEQ ID NO: 39 - DNA, 45 bp, synthetic construct
  source 1..45: mol_type = other DNA
cattatgcggccgcaagcttttattcccactcttgcaggaaacgc

SEQ ID NO: 40 - DNA, 24 bp, synthetic construct
  source 1..24: mol_type = other DNA
ctcataaagctaacccgccgtttt

SEQ ID NO: 41 - DNA, 24 bp, synthetic construct
  source 1..24: mol_type = other DNA
cgttgtcaccagaaaagtgtgacg

SEQ ID NO: 42 - DNA, 53 bp, synthetic construct
  source 1..53: mol_type = other DNA
cacacttttctggtgacaacgtcatttaataatctccagtaaagcctgcacag

SEQ ID NO: 43 - DNA, 34 bp, synthetic construct
  source 1..34: mol_type = other DNA
tactttgttatcgcgatgaatataaactggcact

SEQ ID NO: 44 - DNA, 45 bp, synthetic construct
  source 1..45: mol_type = other DNA
ttgaaatcttcgatgagaaagttttagagctagaaatagcaagtt

SEQ ID NO: 45 - DNA, 45 bp, synthetic construct
  source 1..45: mol_type = other DNA
tttctcatcgaagatttcaaactagtattatacctaggactgagc

SEQ ID NO: 46 - DNA, 27 bp, synthetic construct
  source 1..27: mol_type = other DNA
gtgcagcgttcagaaataagaaaaccc

SEQ ID NO: 47 - DNA, 23 bp, synthetic construct
  source 1..23: mol_type = other DNA
ccaaatggcaaagcccagcatat

(19) United States
(12) Patent Application Publication
(10) Pub. No.: US 2024/0327880 A1
(43) Pub. Date: Oct. 3, 2024

(54) Recombinant Escherichia coli for Producing Chlorogenic Acid and Application Thereof

(71) Applicant: Jiangnan University, Wuxi (CN)

(72) Inventors: Jingwen Zhou, Wuxi (CN); Jian Chen, Wuxi (CN); Lian Wang, Wuxi (CN); Weizhu Zeng, Wuxi (CN)

(21) Appl. No.: 18/437,245

(22) Filed: Feb. 9, 2024

(30) Foreign Application Priority Data
Mar. 31, 2023 (CN) 202310335902X

(51) Int. Cl. C12P 7/62; C12N 9/00; C12N 9/02; C12N 9/04; C12N 9/10; C12N 9/16; C12N 9/88; C12N 9/90; C12N 15/52; C12N 15/70

(52) U.S. Cl. CPC C12P 7/62; C12N 9/0006; C12N 9/0071; C12N 9/10; C12N 9/1085; C12N 9/16; C12N 9/88; C12N 9/90; C12N 9/93; C12N 15/52; C12N 15/70; C12Y 101/01006; C12Y 101/01282; C12Y 114/14009; C12Y 205/01054; C12Y 402/03004; C12Y 403/01023; C12Y 504/99005; C12Y 602/01012

(57) ABSTRACT

The present disclosure provides a recombinant Escherichia coli for producing chlorogenic acid and application thereof. In the present disclosure, tyrosine ammonia-lyase FjTAL derived from Flavobacterium johnsoniae, hpaBC derived from E. coli, 3-deoxy-D-arabino-heptulosonate-7-phosphate synthase mutant aroG^{fbr}, chorismate mutase tyrC derived from Zymomonas mobilis, quinic acid/shikimate-5 dehydrogenase ydiB derived from E. coli, hydroxycinnamoyl CoA:quinic acid transferase NtHQT derived from Nicotiana tabacum, and 4-coumarate:CoA ligase At4CL1 derived from Arabidopsis thaliana are expressed in the recombinant E. coli, thereby constructing a chlorogenic acid biosynthesis pathway in E. coli. Then, the aroB gene and gldA gene derived from E. coli are overexpressed, and an endogenous gene menI is knocked out from the recombinant E. coli. The recombinant strain produced chlorogenic acid by fermentation at a titer of up to 638.2 mg/L in a shake flask or at a titer of 2.8 g/L in a 5-L fermenter.

### CROSS-REFERENCE TO RELATED APPLICATIONS

This application claims the benefit of Chinese patent application No. 202310335902X, filed Mar. 31, 2023, the content of which is incorporated by reference herein.

### SEQUENCE LISTING

This application contains a Sequence Listing file named “WX-2024-01NP Sequence Listing.xml” (75 KB), created on May 31, 2024, the content of which is incorporated by reference herein.

### BACKGROUND OF THE INVENTION

### Field of the Invention

The present disclosure relates to a recombinant E. coli for producing chlorogenic acid and application thereof, belonging to the technical fields of genetic engineering and bioengineering.

### Description of the Related Art

Chlorogenic acid is a natural phenolic acid compound with high content in Eucommia ulmoides, honeysuckle, and green coffee. Structurally, chlorogenic acid is an ester formed by the condensation of one molecule of caffeic acid and one molecule of quinic acid. Chlorogenic acid has good antioxidant, antibacterial, anti-inflammatory, and neuroprotective activities, and has shown great application potential in industries such as health products and pharmaceuticals. In addition, chlorogenic acid can serve as a precursor of chicoric acid, an important phenolic compound in Echinacea purpurea. Therefore, the synthesis of chlorogenic acid has attracted more and more attention from researchers.

At present, the production of chlorogenic acid mainly relies on plant extraction methods, but the production efficiency is affected by factors such as seasonal availability and low plant tissue content. The traditional chemical synthesis method for synthesizing chlorogenic acid involves multi-step reactions with low yields and is not suitable for large-scale production. Therefore, it is desirable to develop biosynthesis methods for producing chlorogenic acid based on metabolic engineering and genetic engineering. At present, previous studies have reported the biosynthesis of chlorogenic acid, which is catalyzed by hydroxycinnamoyl CoA:quinic acid transferase to condense one molecule of caffeoyl-CoA and one molecule of quinic acid. By reconstructing biosynthetic pathways containing hydroxycinnamoyl CoA:quinic acid transferase and caffeoyl-CoA as the core in E. coli and Saccharomyces cerevisiae, microorganisms can achieve the synthesis of chlorogenic acid. However, the flux of caffeic acid and chlorogenic acid synthesis in the chlorogenic acid synthesis pathway is unbalanced, and the two pathways consume many cofactors. Caffeoyl-CoA will be degraded by an endogenous thioesterase, limiting the production of chlorogenic acid.

### SUMMARY OF THE INVENTION

The present disclosure provides a recombinant E. coli for synthesizing chlorogenic acid, expressing hydroxycinnamoyl CoA:quinic acid transferase NtHQT derived from Nicotiana tabacum and 4-coumarate:CoA ligase At4CL1 derived from Arabidopsis thaliana on the basis of an original strain, and the original E. coli strain has the ability to synthesize caffeic acid and quinic acid.

In one embodiment, the original E. coli strain has the ability to synthesize caffeic acid and 3-dehydroquinic acid and expresses quinic acid/shikimate-5 dehydrogenase ydiB derived from E. coli.

In one embodiment, the original E. coli strain is based on E. coli BL21(DE3) ΔtyrR with the tyrR gene being knocked out from the parent E. coli BL21(DE3) strain as described in Wang L et al. (Wang L, Li N, Yu S, Zhou J. Enhancing caffeic acid production in Escherichia coli by engineering the biosynthesis pathway and transporter. Bioresour Technol. 2023; 368:128320).

In one embodiment, the original E. coli strain expresses tyrosine ammonia-lyase FjTAL derived from Flavobacterium johnsoniae and 4-hydroxyphenylacetate-3-monooxygenase hpaBC derived from E. coli to construct a biosynthetic module of caffeic acid, expresses 3-deoxy-D-arabino-heptulosonate-7-phosphate synthase mutant aroG^{fbr} and chorismate mutase tyrC derived from Zymomonas mobilis to relieve feedback inhibition of L-tyrosine synthesis, and expresses quinic acid/shikimate-5 dehydrogenase ydiB derived from E. coli.

In one embodiment, the recombinant E. coli further overexpresses 3-dehydroquinate synthase aroB and glycerol dehydrogenase gldA in the endogenous shikimate pathway of E. coli to increase the flux of shikimate and the level of intracellular cofactor NADH, respectively.

In one embodiment, the recombinant E. coli further knocks out endogenous thioesterase MenI, improving the ability of E. coli to synthesize chlorogenic acid.

In one embodiment, the Genbank accession number of tyrosine ammonia-lyase is WP_012023194.1 (SEQ ID NO:12), the Genbank accession numbers of hpaBC are WP_000801472.1 (SEQ ID NO:13) and WP_001175451.1 (SEQ ID NO:14), the Genbank accession number of quinic acid/shikimate-5 dehydrogenase is WP_140023115.1 (SEQ ID NO:15), the Genbank accession number of hydroxycinnamoyl CoA:quinic acid transferase is AJ582651.1 (SEQ ID NO:16), and the Genbank accession number of 4-coumarate:CoA ligase 4CL is NP_175579.1 (SEQ ID NO:17).

In one embodiment, the aroG mutant aroG^{fbr} contains an amino acid sequence shown in a Genbank accession number: AXN70009.1 (SEQ ID NO:18), which contains the mutation to relieve feedback inhibition of L-phenylalanine.

In one embodiment, the chorismate mutase tyrC derived from Z. mobilis contains an amino acid sequence shown in a Genbank accession number: AAA27684.1 (SEQ ID NO:19).

In one embodiment, the recombinant E. coli expresses 3-dehydroquinate synthase aroB (Genbank accession number: HBP1284526.1, SEQ ID NO:20) derived from E. coli, increases the flux of the shikimate pathway and significantly increases the titer of chlorogenic acid.

In one embodiment, the recombinant E. coli expresses 3-dehydroquinate synthase aroB (Genbank accession number: HBP1284526.1, SEQ ID NO:20) derived from E. coli, and expresses glycerol dehydrogenase gldA (Genbank accession number: WP_251286818.1, SEQ ID NO:21) derived from E. coli, increases the flux of the shikimate pathway and the level of intracellular NADH.

In one embodiment, the nucleotide sequences of the gene FjTAL, gene hpaBC, gene ydiB, gene NtHQT, and gene At4CL1 are shown in SEQ ID NO:1, SEQ ID NO:2, SEQ ID NO:3, SEQ ID NO:4, and SEQ ID NO:5, respectively.

In one embodiment, a sequence of aroG^{fbr} gene is shown in SEQ ID NO:6.

In one embodiment, a sequence of tyrC gene is shown in SEQ ID NO:7.

In one embodiment, a sequence of aroB gene is shown in SEQ ID NO:8.

In one embodiment, a sequence of gldA gene is shown in SEQ ID NO:9.

In one embodiment, pETDuet-1 is used as an expression vector expressing the aroG^{fbr} gene, the tyrC gene, the ydiB gene, and the NtHQT gene.

In one embodiment, pACYCDuet-1 is used as an expression vector expressing the FjTAL gene, the hpaBC gene and the At4CL1 gene.

In one embodiment, pCDFDuet-1 is used as an expression vector expressing the aroB gene and the gldA gene.

The present disclosure also provides the application of recombinant E. coli for producing chlorogenic acid or derivatives thereof by fermentation.

The present disclosure provides a method for producing chlorogenic acid, comprising using the recombinant E. coli to produce chlorogenic acid by fermentation.

In one embodiment, the recombinant E. coli is inoculated into a fermentation system and cultured at 37° C. for 3-4 h, IPTG with a final concentration of 0.1-0.2 mM is added, the chlorogenic acid is synthetized at 30° C. and 200-220 r/min, and fermentation is carried out for 24-72 h.

In one embodiment, the fermentation system contains glucose, glycerol, (NH₄)₂SO₄, K₂HPO₄·3H₂O, KH₂PO₄, MgSO₄·7H₂O, citric acid, vitamin B1, yeast extract, ascorbic acid, and betaine.

The present disclosure provides the application of the recombinant E. coli for producing chlorogenic acid or derivatives thereof.

In one embodiment, the derivatives include but are not limited to chicoric acid, methyl chlorogenic acid, and ethyl chlorogenic acid.

### Beneficial Effects

In the present disclosure, the E. coli BL21(DE3) ΔtyrR is used as a host, and tyrosine ammonia-lyase FjTAL derived from F. johnsoniae, hpaBC derived from E. coli BL21(DE3), 3-deoxy-D-arabino-heptulosonate-7-phosphate synthase mutant aroG^{fbr}, chorismate mutase tyrC derived from Z. mobilis, quinic acid/shikimate-5 dehydrogenase ydiB derived from E. coli, hydroxycinnamoyl CoA:quinic acid transferase NtHQT derived from N. tabacum, and 4-coumarate:CoA ligase At4CL1 derived from A. thaliana are expressed in the host strain, achieving biosynthesis of chlorogenic acid in the recombinant E. coli. Then the aroB gene derived from E. coli are overexpressed, increasing the flux of the shikimate pathway. The gldA gene is overexpressed, increasing the supply of cofactor NADH. The endogenous thioesterase gene menI is knocked out, increasing the intracellular stability of caffeoyl-CoA, thereby increasing the content and stability of the precursor caffeoyl-CoA of chlorogenic acid. The accumulation of chlorogenic acid can reach 638.2 mg/L after fermentation in a shake flask for 48 h, or 2.8 g/L in a 5-L fermenter. The present disclosure constructs a heterologous plant biosynthetic pathway in E. coli, providing a new biosynthesis approach for efficient production of chlorogenic acid and derivatives thereof.

### BRIEF DESCRIPTION OF DRAWINGS

FIG. 1 is a schematic metabolic diagram of heterologous biosynthesis of chlorogenic acid in E. coli.

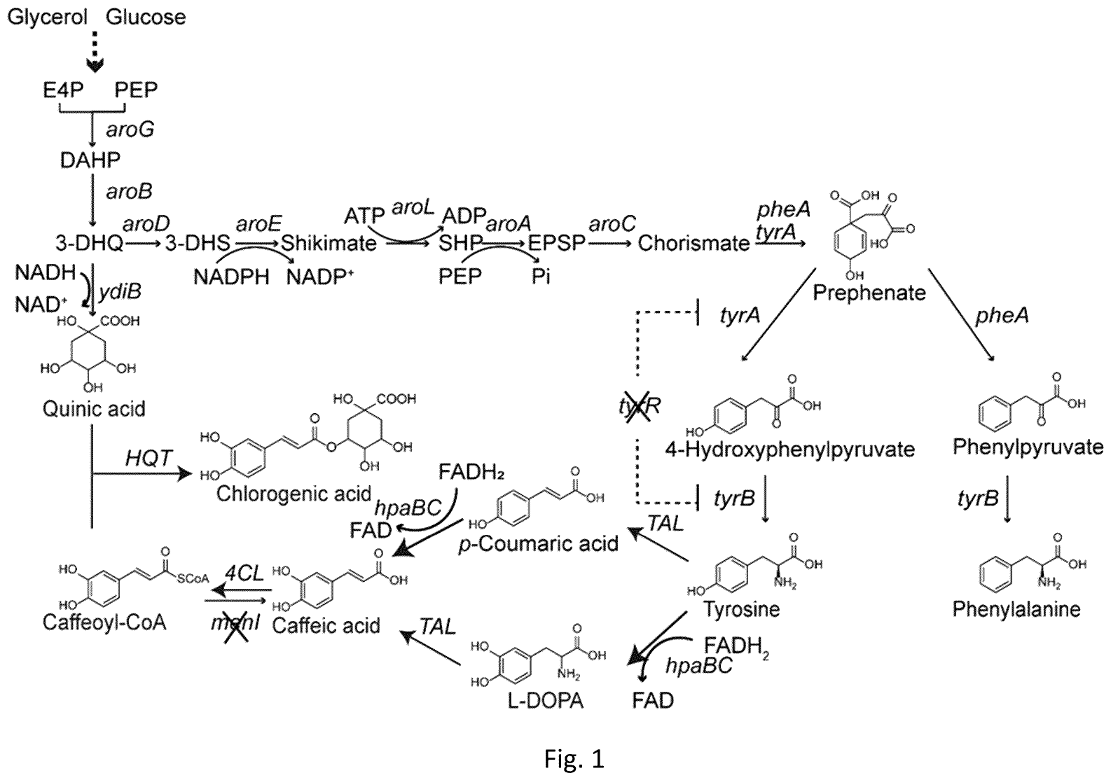

FIG. 2A is a chromatogram of chlorogenic acid detection of E. coli CGA30 cultured in a fermentation medium.

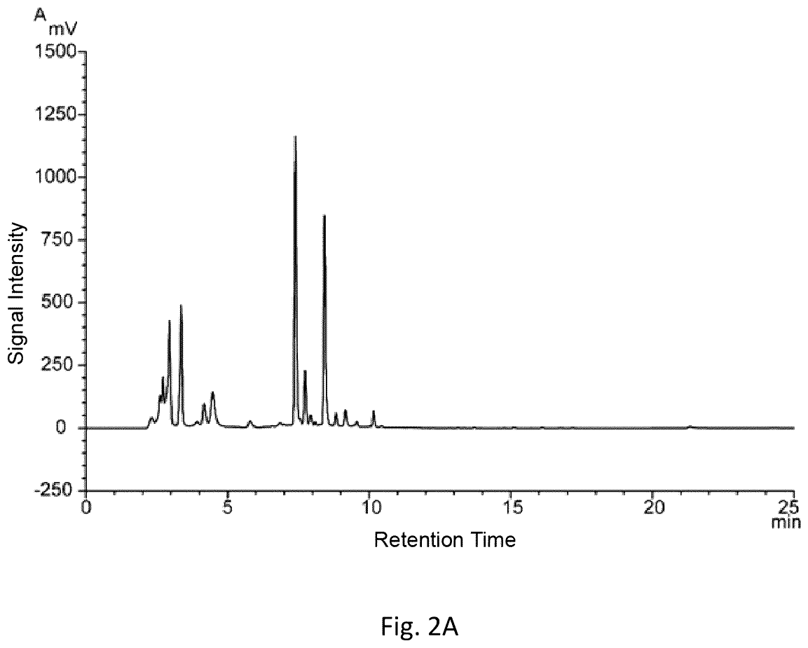

FIG. 2B is a chromatogram of caffeic acid standard.

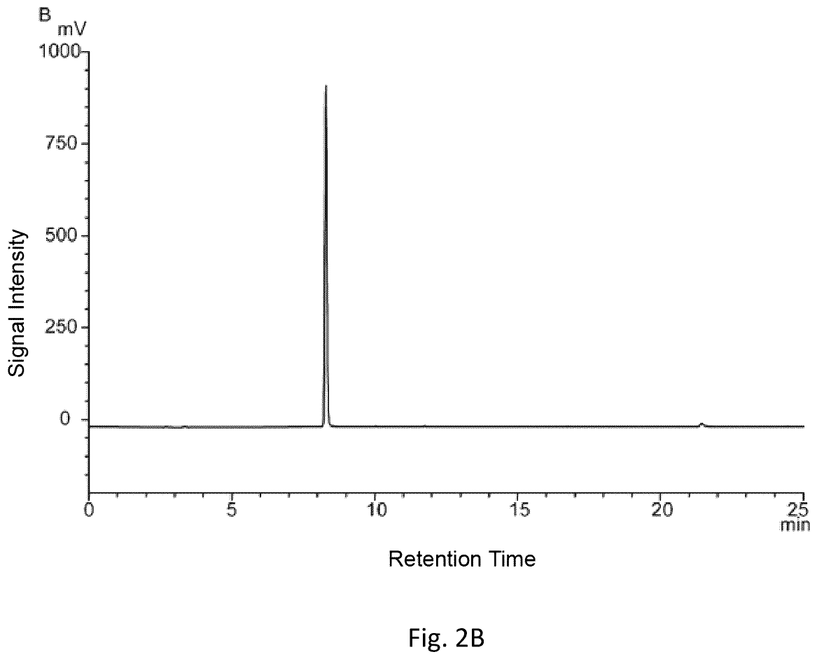

FIG. 2C is a chromatogram of chlorogenic acid standard.

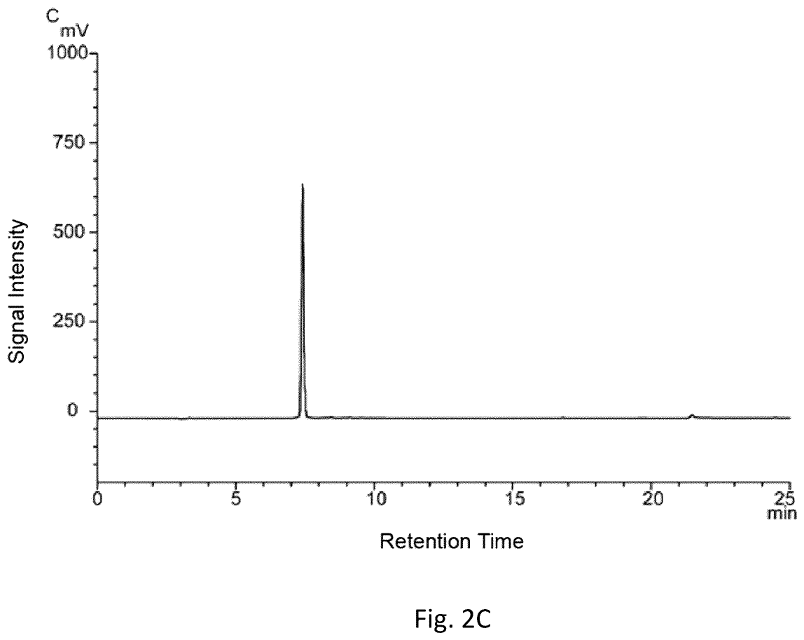

FIG. 3 is a chlorogenic acid titer diagram of different recombinant E. coli cultured in a fermentation medium.

FIG. 4 shows the biosynthesis of chlorogenic acid from an optimized strain CGA30 in a 5-L fermenter.

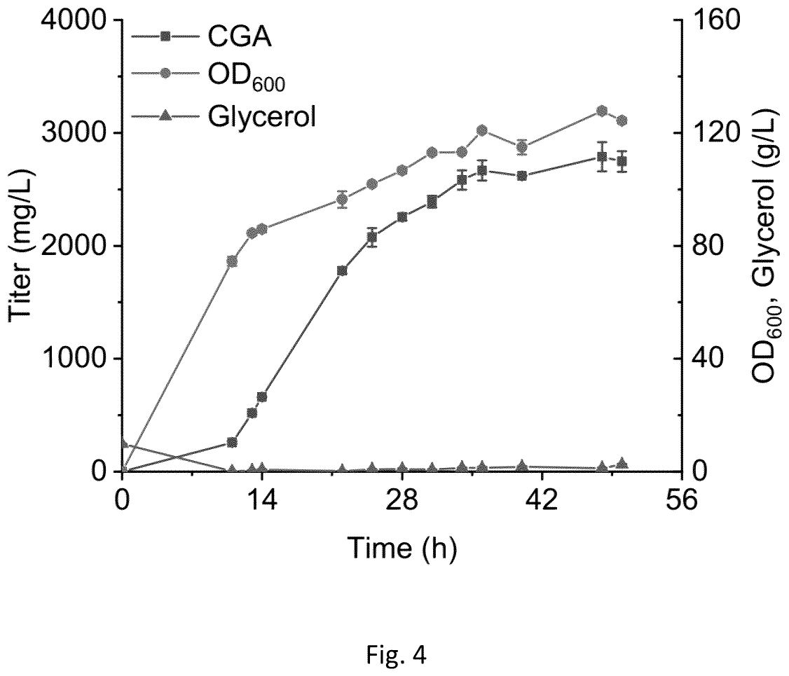

### DETAILED DESCRIPTION OF THE INVENTION

(I) Media

Seed medium (LB): peptone 10 g/L, yeast extract 5 g/L, and sodium chloride 5 g/L; and 2% (mass fraction) agar powder added to the solid medium.

Fermentation medium: glucose 25 g/L, glycerol 10 g/L, (NH₄)₂SO₄ 7.5 g/L, K₂HPO₄·3H₂O 3 g/L, KH₂PO₄ 2 g/L, MgSO₄·7H₂O 2 g/L, ascorbic acid 0.45 g/L, sodium citrate 1 g/L, vitamin B1 0.1 g/L, yeast extract 7 g/L, and betaine 5 g/L. 250 g/L glucose was separated and sterilized separately, and mixed well before inoculation.

Supplement medium: glycerol 500 g/L, yeast extract 10 g/L, MgSO₄·7H₂O 3 g/L, and ascorbic acid 0.45 g/L.

(II) PCR reaction system and amplification conditions: forward primer (10 μM) 1 μL, reverse primer (10 μM) 1 μL, template DNA 10-50 ng, 2×Phanta Max Master Mix 25 μL and double distilled water added to 50 μL. Amplification conditions: pre-denaturation at 95° C. for 3 min; 30 cycles (95° C. for 15 s, 55° C. for 15 s, and 72° C. for 15 s), and extension at 72° C. for 5 min.

(III) Preparation of E. coli competent cells: a glycerol tube of E. coli JM109 was streaked on a corresponding LB plate, and cultured at 37° C. overnight (for about 12 h). After 12 h, monoclonal cells were picked and inoculated into a 50 mL shaking flask containing 5 mL of LB medium, and then cultured at 37° C. at 220 r/min until OD₆₀₀=0.6. The bacterial solution was transferred to a 50 mL centrifuge tube, and placed on ice for about 15 min. The centrifuge tube was centrifuged at 4000 r/min at 4° C. for 5 min, and the supernatant was removed. 5 mL of solution A was added for resuspension. The centrifuge tube was centrifuged at 4000 r/min at 4° C. for 5 min, and the supernatant was removed. 5 mL of solution B was added to resuspend the cells, divided according to 100 μL/package, and stored at −80° C.

(IV) Transformation of E. coli: the E. coli competent cells were thawed on ice. 10 μL of recombinant product (plasmid 50 ng) was added into 100 μL of competent cells, evenly mixed by flicking, and allowed to stand on ice for 30 min. The competent cells were subjected to heat shock in a 42° C. water bath for 45 s, and allowed to stand on ice for 2 min. 1 mL of LB medium was added, and the cells were shaken at 37° C. at 220 r/min for 60 min. The bacterial solution was centrifuged at 4500 r/min for 2 min, and 900 μL of supernatant was removed. The cells were resuspended with the remaining medium, and then the bacterial solution was coated on a resistant plate.

(V) Determination of chlorogenic acid by HPLC: after the fermentation was completed, 500 μL of fermentation broth was added to the same volume of methanol, and then shaken vigorously and mixed uniformly. The mixture was centrifuged at 14000 r/min for 10 min. The supernatant was filtered through a 0.22 μm organic phase filter membrane, and a Shimadzu LC-20A high-performance liquid chromatograph was used to analyze the product profile. A Thermo Fisher C18 column (4.6 mm×250 mm, 5 μm) was used for chromatographic separation. The temperature of the column oven was set to 40° C. The injection volume was 10 μL. The mobile phases were: phase A: ultrapure water (with 0.1% trifluoroacetic acid), and phase B: acetonitrile (with 0.1% trifluoroacetic acid). The total flow rate was 1 mL/min. The type of elution was gradient elution: 0-10 min, phase B: 10-60%; 10-20 min, phase B: 60-80%; 20-22 min, phase B: 80-10%; 22-25 min, phase B: 10%. The detector wavelength was 323 nm for products.

(VI) The information of strains is shown in Table 1:

TABLE 1
Strains and genotypes involved in the present disclosure

| Strain Name | Genotype |
|---|---|
| E. coli BL21(DE3) | E. coli BL21(DE3) with tyrR knocked out |
| ΔtyrR | |
| E. coli BL21(DE3) | E. coli BL21(DE3) with tyrR and menI knocked out |
| ΔtyrRΔmenI | |
| CGA01 | E. coli BL21(DE3) ΔtyrR containing plasmids pETDuet-aroG^{fbr}-tyrC- |
| | ydiB-NtHQT and pACYCDuet-FjTAL-RBS-HpaBC-At4CL1 |
| CGA01C | E. coli BL21(DE3) ΔtyrR containing plasmids pETDuet-aroG^{fbr}-tyrC- |
| | ydiB-CsHQT and pACYCDuet-FjTAL-RBS-HpaBC-At4CL1 |
| CGA03 | E. coli BL21(DE3) ΔtyrR containing plasmids pETDuet-aroG^{fbr}-tyrC- |
| | ydiB-EpHQT and pACYCDuet-FjTAL-RBS-HpaBC-At4CL1 |
| CGA06 | E. coli BL21(DE3) ΔtyrR containing plasmids pETDuet-aroG^{fbr}-tyrC- |
| | ydiB-NtHQT, pACYCDuet-FjTAL-RBS-HpaBC-At4CL1 and pCDF- |
| | aroB |
| CGA22 | E. coli BL21(DE3) ΔtyrR containing plasmids pETDuet-aroG^{fbr}-tyrC- |
| | ydiB-NtHQT, pACYCDuet-FjTAL-RBS-HpaBC-At4CL1 and pCDF- |
| | aroB-gldA |
| CGA30 | E. coli BL21(DE3) ΔtyrRΔmenI containing plasmids pETDuet- |
| | aroG^{fbr}-tyrC-ydiB-NtHQT, pACYCDuet-FjTAL-RBS-HpaBC- |
| | At4CL1 and pCDF-aroB-gldA |

### Example 1. Construction of Recombinant E. coli for Synthesizing Chlorogenic Acid

E. coli BL21(DE3) ΔtyrR (the strain with ΔtyrR knocked out on the basis of E. coli BL21(DE3), Wang L, Li N, Yu S, Zhou J. Enhancing caffeic acid production in Escherichia coli by engineering the biosynthesis pathway and transporter. Bioresour Technol. 2023; 368:128320) was used as an original strain for synthesizing chlorogenic acid. The synthesis pathways of chlorogenic acid include the synthesis of caffeoyl CoA and quinic acid, which are substrates used for production of chlorogenic acid. Previously constructed plasmid pACYCDuet-FjTAL-RBS-HpaBC-At4CL1 (nucleotide sequence as shown in SEQ ID NO:22) (Wang L, Wang H, Chen J, Qin Z, Yu S, Zhou J. Coordinating caffeic acid and salvianic acid A pathways for efficient production of rosmarinic acid in Escherichia coli. Metab Eng. 2023; 76:29-38) was transformed into E. coli BL21(DE3) ΔtyrR to construct a biosynthesis pathway for caffeoyl CoA in the E. coli cells.

To construct a biosynthetic pathway of quinic acid and chlorogenic acid, gene sequences for key enzymes of the biosynthetic pathway are generated using chemical synthesis or PCR amplification. Using the genomic DNA of E. coli BL21(DE3) as a template, the ydiB fragment (nucleotide sequence as shown in SEQ ID NO:3) was amplified by using primer pair F1/R1. Using the synthesized NtHQT sequence (nucleotide sequence as shown in SEQ ID NO:4) as a template, the NtHQT fragment was amplified by using primer pair F2/R2 by PCR. Using the plasmid pETDuet-aroG^{fbr}-tyrC (SEQ ID NO:23) (Wang L, Li N, Yu S, Zhou J. Enhancing caffeic acid production in Escherichia coli by engineering the biosynthesis pathway and transporter. Bioresour Technol. 2023; 368:128320) as a template, amplification was carried out by using primer pair F3/R3, and the product was purified to obtain a pETDuet-aroG^{frb}-tyrC skeleton fragment. The ydiB fragment, NtHQT fragment, and pETDuet-aroG^{fbr}-tyrC skeleton fragment were recombined by Gibson assembly to obtain a recombinant vector, and the recombinant vector was transformed into E. coli JM109. Plasmid was extracted and sequenced to verify that a correct recombinant vector pETDuet-aroG^{fbr}-tyrC-ydiB-NtHQT was obtained.

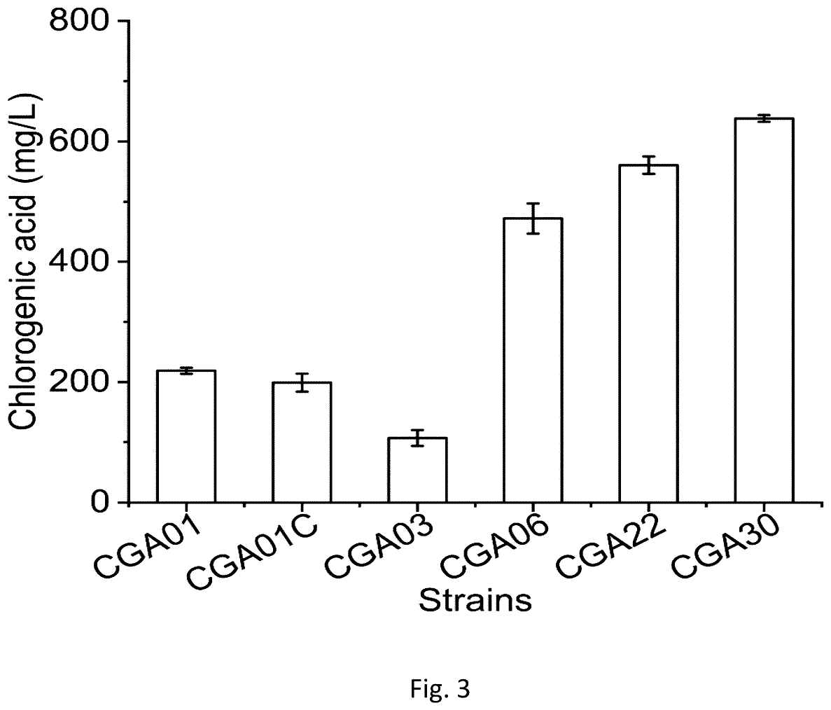

The recombinant vectors pACYCDuet-FjTAL-RBS-HpaBC-At4CL1 (RBS sequence: AAGGAGATATACC) and pETDuet-aroG^{fbr}-tyrC-ydiB-NtHQT were transformed into E. coli BL21(DE3) ΔtyrR to obtain strain CGA01. The engineering strain was activated and cultured in the seed medium at 37° C. at 220 r/min for 12 h to obtain a seed solution, and the seed solution was inoculated into a fermentation medium containing 100 μg/mL (final concentration) ampicillin and 37 μg/mL chloramphenicol according to an inoculation amount of 2%. After 3 h of culture at 37° C. at 220 r/min (OD₆₀₀ reached 0.6-0.8), IPTG with a final concentration of 0.1 mM was added, and induction and fermentation were carried out at 30° C. at 220 r/min for 48 h to synthesize chlorogenic acid. As shown in FIG. 3, in the fermentation broth of the strain CGA01, the titer of chlorogenic acid was 218.9 mg/L.

In order to compare hydroxycinnamoyl CoA:quinic acid transferase derived from different sources, using the synthesized EpHQT fragment from E. purpurea as a template (nucleotide sequence as shown in SEQ ID NO:10), PCR amplification was carried out by using primer pair F4/R4, and the product was purified to obtain an EpHQT fragment. The ydiB fragment, EpHQT fragment, and pETDuet-aroG^{fbr}-tyrC skeleton fragment were recombined by Gibson assembly to obtain a recombinant vector, and the recombinant vector was transformed into E. coli JM109. Plasmid extraction was carried out, and sequencing was carried out to verify that a correct recombinant vector pETDuet-aroG^{fbr}-tyrC-ydiB-EpHQT was obtained. The recombinant vectors pACYCDuet-FjTAL-RBS-HpaBC-At4CL1 and pETDuet-aroG^{fbr}-tyrC-ydiB-EpHQT were transformed into E. coli BL21(DE3) ΔtyrR to obtain an engineering strain CGA03. The engineering strain was activated and cultured in the seed medium at 37° C. at 220 r/min for 12 h to obtain a seed solution, and the seed solution was inoculated into a fermentation medium containing 100 μg/mL (final concentration) ampicillin and 37 μg/mL chloramphenicol according to an inoculation amount of 2%. After 3 h of culture at 37° C. at 220 r/min until OD₆₀₀ reached 0.6-0.8, IPTG with a final concentration of 0.1 mM was added, and induction and fermentation were carried out at 30° C. at 220 r/min for 48 h to synthesize chlorogenic acid. As shown in FIG. 3, in the fermentation broth of the strain CGA03, the titer of chlorogenic acid was 106.7 mg/L.

All primer sequences are listed in Table 2.

TABLE 2
Primer sequences

| Primer | | |
|---|---|---|
| Name | Primer Sequence | SEQ ID Number |
| F1 | AAGTATAAGAAGGAGATATACATATGGATGTTACCG | SEQ ID NO: 24 |
| | CAAAATACGAATTGAT | |
| R1 | TCCTTTCAGGCACCGAACCCCATGA | SEQ ID NO: 25 |
| F2 | GGTTCGGTGCCTGAAAGGAGATATACCATGGGCTCT | SEQ ID NO: 26 |
| | GAAAAAATGATGAAAATCAACAT | |
| R2 | GTGGCAGCAGCCTAGGTTAATTAGAATTCGTACAGG | SEQ ID NO: 27 |
| | TATTTTTCAAACAGCGG | |
| F3 | TTAACCTAGGCTGCTGCCAC | SEQ ID NO: 28 |
| R3 | CATATGTATATCTCCTTCTTATACTTAACTAATATAC | SEQ ID NO: 29 |
| | TAAGATGG | |
| F4 | GTTCGGTGCCTGAAAGGAGATATACCATGAACATTA | SEQ ID NO: 30 |
| | CCATCACGAAATCCTCTCTG | |
| R4 | GTGGCAGCAGCCTAGGTTAATTAGAATTCATACAGA | SEQ ID NO: 31 |
| | TATTTTTCAAACAGCGGC | |

### Example 2. Overexpression of aroB to Improve Flux of Shikimate

In order to improve flux of shikimate, aroB gene was overexpressed in the engineering strain. Using the genome DNA of E. coli BL21(DE3) as a template, the aroB fragment (nucleotide sequence as shown in SEQ ID NO:8) was amplified by using primer pair F5/R5. Using pCDFDuet-1 vector as a template, amplification was carried out by using primer pair F6/R6, and the product was purified to obtain a pETDuet-1 skeleton fragment. The aroB fragment and the vector pETDuet-1 skeleton fragment were recombined by Gibson assembly to obtain a recombinant vector, and the recombinant vector was transformed into E. coli JM109. Plasmid extraction was carried out, and sequencing was carried out to verify that a correct recombinant vector pETDuet-aroB was obtained. The recombinant vector pCDFDuet-aroB was transformed into the strain CGA01 constructed in Example 1 to obtain an engineering strain CGA06. The engineering strain CGA06 was activated and cultured in the seed medium at 37° C. at 220 r/min for 12 h to obtain a seed solution, and the seed solution was inoculated into a fermentation medium containing 100 μg/mL (final concentration) ampicillin, 50 μg/mL spectinomycin and 37 μg/mL chloramphenicol according to an inoculation amount of 2%. After 3 h of culture at 37° C. at 220 r/min until OD₆₀₀ reached 0.6-0.8, IPTG with a final concentration of 0.1 mM was added, and induction and fermentation were carried out at 30° C. at 220 r/min for 48 h to synthesize chlorogenic acid. As shown in FIG. 3, in the fermentation broth of CGA06, the titer of chlorogenic acid was 472.0 mg/L.

All primer sequences are listed in Table 3.

TABLE 3
Primer sequences

| Primer | | |
|---|---|---|
| Name | Primer Sequence | SEQ ID Number |
| F5 | TAAGGAGATATACCATGGGCATGGAGAGGATTGTCG | SEQ ID NO: 32 |
| | TTACTCTCGG | |
| R5 | CATTATGCGGCCGCAAGCTTTTACGCTGATTGACAAT | SEQ ID NO: 33 |
| | CGGCAATG | |
| F6 | AAGCTTGCGGCCGCATAATGCT | SEQ ID NO: 34 |
| R6 | GCCCATGGTATATCTCCTTATTAAAGTTAAAC | SEQ ID NO: 35 |

### Example 3. Overexpression of gldA to Improve Regeneration of Intracellular Cofactor

In order to increase the level of intracellular cofactor NADH, gene gldA was overexpressed in the engineering strain above. Using the genome DNA of E. coli BL21(DE3) as a template, the aroB fragment (nucleotide sequence as shown in SEQ ID NO:8) was amplified by using primer pair F7/R7. Using the genome DNA of E. coli BL21(DE3) as a template, the gldA fragment (nucleotide sequence as shown in SEQ ID NO:9) was amplified by using primer pair F8/R8. The aroB fragment, the gldA fragment and the vector pETDuet-1 skeleton in Example 2 were recombined by Gibson assembly to obtain a recombinant vector, and the recombinant vector was transformed into E. coli JM109. Plasmid extraction was carried out, and sequencing was carried out to verify that a correct recombinant vector pCDF-aroB-gldA was obtained. The recombinant vector pCDF-aroB-gldA was transformed into the strain CGA01 constructed in Example 1 to obtain an engineering strain CGA22. The engineering strain CGA22 was activated and cultured in the seed medium at 37° C. at 220 r/min for 12 h to obtain a seed solution, and the seed solution was inoculated into a fermentation medium containing 100 μg/mL (final concentration) ampicillin, 50 μg/mL spectinomycin and 37 μg/mL chloramphenicol according to an inoculation amount of 2%. After 3 h of culture at 37° C. at 220 r/min until OD₆₀₀ reached 0.6-0.8, IPTG with a final concentration of 0.1 mM was added, and induction and fermentation were carried out at 30° C. at 220 r/min for 48 h to synthesize chlorogenic acid. As shown in FIG. 3, in the fermentation broth of CGA22, the titer of chlorogenic acid was 560.6 mg/L.

All primer sequences are listed in Table 4.

TABLE 4
Primer sequences

| Primer | | |
|---|---|---|
| Name | Primer Sequence | SEQ ID Number |
| F7 | TAAGGAGATATACCATGGGCATGGAGAGGATTGTCG | SEQ ID NO: 36 |
| | TTACTCTCGG | |
| R7 | CTCCTTTTACGCTGATTGACAATCGGCAATG | SEQ ID NO: 37 |
| F8 | CCGATTGTCAATCAGCGTAAAAGGAGATATACCATG | SEQ ID NO: 38 |
| | GACCGCATTATTCAATCACCG | |
| R8 | CATTATGCGGCCGCAAGCTTTTATTCCCACTCTTGCA | SEQ ID NO: 39 |
| | GGAAACGC | |

### Example 4. Optimization of Stability of Caffeoyl CoA

In order to improve stability of caffeoyl CoA and reduce the conversion of caffeoyl CoA into caffeic acid, the endogenous thioesterase-encoding gene menI in E. coli was knocked out. Using the E. coli BL21(DE3) genome as a template, an upstream homologous arm U1 and a downstream homologous arm D1 of the gene menI were amplified respectively by using primer pairs F9/R9 and F10/R10, and the fragments were purified. Using the purified fragments U1 and D1 as templates, amplification was carried out by using a primer pair F9/R10 to obtain a knockout kit UD1, and the fragment was purified. In order to obtain pTarget-menI for knocking out menI, using pTarget as a template, amplification was carried out by using a primer pair F11/R11, and the fragment was purified. The purified fragment was transformed into E. coli JM109. Plasmid was extracted and sequenced to verify that a correct recombinant vector pTarget-menI was obtained.

In order to produce pCas9-containing E. coli BL21(DE3) ΔtyrR electroporation-competent cells, the pCas9 plasmid was transformed into E. coli BL21(DE3) ΔtyrR chemically competent cells. The transformed monoclonal cells were picked and inoculated into 4 mL of LB medium, kanamycin with a final concentration of 50 μg/mL was added, and the cells were cultured at 30° C. for 12 h. The bacterial solution was inoculated into 50 mL of LB medium according to an inoculation amount of 2%, and a kanamycin solution with a final concentration of 50 μg/mL and 10 mM arabinose were added. The cells were cultured at 30° C. at 220 r/min for 4 h-6 h until OD reached 0.6. The bacterial solution was transferred into a 50 ml centrifuge tube, and allowed to stand on ice for 15 min. The centrifuge tube was centrifuged at 4000 r/min at 4° C. for 10 min, and the supernatant was removed. 10 mL of 10% glycerol was added for resuspension. The operation was repeated twice, and the bacterial solution was divided according to 100 μL/package, and stored at −80° C. 400 ng of recombinant vector pTarget-menI and 1200 ng of knockout kit UD1 were added to the E. coli BL21(DE3) ΔtyrR electroporation-competent cells. The suspension was allowed to stand on ice for 10 min, and then transferred into a 1 mm electroporation cuvette that had been precooled for 10 min, and electroporation was carried out under a voltage of 1.8 kv. After the electroporation was completed, 1 ml of LB liquid medium was added, and the cells were cultured at 30° C. for 1.5 h. Colony PCR was carried out by using a primer pair F12/R12 for verification. pTarget-menI and pCas9 were dropped out from the correct monoclonal cells according to the method in the literature (disclosed in the paper: Jiang Y, Chen B, Duan C, Sun B, Yang J, Yang S. Multigene editing in the Escherichia coli genome via the CRISPR-Cas9 system. Appl Environ Microbiol. 2015; 81(7):2506-2514) to obtain an engineering strain E. coli BL21(DE3) ΔtyrRΔmenI. The recombinant vectors pACYCDuet-FjTAL-RBS-HpaBC-At4CL1, and pETDuet-aroG^{fbr}-tyrC-ydiB-NtHQT constructed in Example 1 and pCDF-aroB-gldA constructed in Example 3 were transformed into the E. coli BL21(DE3) ΔtyrRΔmenI to obtain an engineering strain CGA30. The engineering strain CGA30 was activated and cultured in the seed medium at 37° C. at 220 r/min for 12 h to obtain a seed solution, and the seed solution was inoculated into a fermentation medium containing 100 μg/mL (final concentration) ampicillin, 50 μg/mL spectinomycin and 37 μg/mL chloramphenicol according to an inoculation amount of 2%. After 3 h of culture at 37° C. at 220 r/min (until OD₆₀₀ reached 0.6-0.8), IPTG with a final concentration of 0.1 mM was added, and induction and fermentation were carried out at 30° C. at 220 r/min for 48 h to synthesize chlorogenic acid. As shown in FIG. 3, in the fermentation broth of CGA30, the titer of chlorogenic acid was 638.2 mg/L.

All primer sequences are listed in Table 5.

TABLE 5
Primer sequences

| Primer | | |
|---|---|---|
| Name | Primer Sequence | SEQ ID Number |
| F9 | CTCATAAAGCTAACCCGCCGTTTT | SEQ ID NO: 40 |
| R9 | CGTTGTCACCAGAAAAGTGTGACG | SEQ ID NO: 41 |
| F10 | CACACTTTTCTGGTGACAACGTCATTTAATAATCTCCAGTAAAGCC | SEQ ID NO: 42 |
| | TGCACAG | |
| R10 | TACTTTGTTATCGCGATGAATATAAACTGGCACT | SEQ ID NO: 43 |
| F11 | TTGAAATCTTCGATGAGAAAGTTTTAGAGCTAGAAATAGCAAGTT | SEQ ID NO: 44 |
| R11 | TTTCTCATCGAAGATTTCAAACTAGTATTATACCTAGGACTGAGC | SEQ ID NO: 45 |
| F12 | GTGCAGCGTTCAGAAATAAGAAAACCC | SEQ ID NO: 46 |
| R12 | CCAAATGGCAAAGCCCAGCATAT | SEQ ID NO: 47 |

### Example 5. Optimization of Titer of Chlorogenic Acid in a 5-L Fermenter

The strain CGA30 constructed in Example 4 was applied to a 5-L fermenter for fed-batch fermentation. The engineering strain CGA30 was activated and cultured in the seed medium at 37° C. and 220 r/min for 8 h to obtain a primary seed solution. Then, the primary seed solution was inoculated into fresh seed medium according to an inoculation amount of 8% and cultured at 37° C. and 220 r/min for 6 h to obtain a secondary seed solution. The secondary seed solution was inoculated into a 5-L fermenter with a 2.5 L fermentation medium (containing 100 μg/mL (final concentration) ampicillin, 50 μg/mL spectinomycin and 37 μg/mL chloramphenicol) according to an inoculation amount of 6%. The initial temperature of the fermenter was set to 37° C., the initial stirring blade speed was set to 300 r/min, and the ventilation rate was set to 2 L/min. In order to maintain the dissolved oxygen 30% or above, the stirring speed was controlled at 300-800 r/min. The pH was controlled at 6.7±0.1 by automatically adding 100% ammonia. When the OD₆₀₀ reached around 40, the temperature of the fermenter was decreased to 30° C. and 0.1 mM IPTG was added for induction. When the dissolved oxygen began to rebound after 11 h of fermentation (at which point the OD₆₀₀ reached 74.6), the supplement medium was added to the fermenter at a flow rate of 10 mL/h. In order to increase stability of chlorogenic acid in the fermentation process, the pH was decreased to 6.0±0.1 after 24 h of fermentation. The titer of chlorogenic acid of strain CGA30 reached 2.8 g/L, and the OD₆₀₀ at this time reached 127.8 at 48 h.

### Comparative Example 1

The specific embodiment is the same as Example 1 except that NtHQT was replaced with CsHQT (nucleotide sequence as shown in SEQ ID NO:11) derived from Cynara scorymus, and a recombinant strain CGA01C containing the recombinant plasmid pETDuet-aroG^{fbr}-tyrC-ydiB-CsHQT and pACYCDuet-FjTAL-RBS-HpaBC-At4CL1 was constructed. The results show that the titer of chlorogenic acid is only 199.1 mg/L, which is significantly less than that of the strain CGA01 at 48 h.

While the present invention has been described in some embodiments for purposes of clarity and understanding, it is not intended to limit the scope of the invention. One skilled in the art will appreciate that various changes in form and detail can be made without departing from the true scope of the invention. The true scope of the present invention shall only be as defined in the Claims.

What is claimed is:

1. A recombinant Escherichia coli (E. coli) for synthesizing chlorogenic acid, wherein the recombinant E. coli is based on an original E. coli strain that is transformed with hydroxycinnamoyl CoA:quinic acid transferase derived from Nicotiana tabacum and 4-coumarate:CoA ligase derived from Arabidopsis thaliana, wherein the original E. coli strain has the ability to synthesize caffeic acid and quinic acid.

2. The recombinant E. coli of claim 1, wherein the original E. coli strain can synthesize caffeic acid and 3-dehydroquinic acid and expresses quinic acid/shikimate-5 dehydrogenase derived from E. coli.

3. The recombinant E. coli of claim 2, wherein the original E. coli strain is based on E. coli BL21(DE3) ΔtyrR that is transformed with tyrosine ammonia-lyase FjTAL derived from Flavobacterium johnsoniae, 4-hydroxyphenylacetate-3-monooxygenase hpaBC derived from E. coli, 3-deoxy-D-arabino-heptulosonate-7-phosphate synthase mutant aroG^{fbr} and chorismate mutase tyrC derived from Zymomonas mobilis, and quinic acid/shikimate-5 dehydrogenase ydiB derived from E. coli.

4. The recombinant E. coli of claim 3, wherein the recombinant E. coli further overexpresses 3-dehydroquinate synthase aroB and glycerol dehydrogenase gldA in the endogenous shikimate pathway of E. coli.

5. The recombinant E. coli of claim 4, wherein thioesterase menI is knocked out in the recombinant E. coli.

6. The recombinant E. coli of claim 1, wherein the hydroxycinnamoyl CoA:quinic acid transferase contains an amino acid sequence shown in SEQ ID NO:16, and the 4-coumarate:CoA ligase 4CL contains an amino acid sequence shown in SEQ ID NO:17.

7. The recombinant E. coli of claim 5, wherein pETDuet-1 is used as an expression vector expressing aroG^{fbr} gene, tyrC gene, ydiB gene, and NtHQT gene; and/or
pACYCDuet-1 is used as an expression vector expressing FjTAL gene, hpaBC gene and At4CL1 gene; and/or pCDFDuet-1 is used as an expression vector expressing aroB gene and gldA gene.

8. A method for producing chlorogenic acid by fermentation, wherein the recombinant E. coli of claim 1 is cultured in a medium for a period of time, 0.1 mM IPTG is added, and fermentation is carried out for 24-72 h.

9. The method of claim 8, wherein the recombinant E. coli is inoculated into a fermentation system and cultured at 37° C. for 3-4 h, IPTG with a final concentration of 0.1-0.2 mM is added, the chlorogenic acid is induced and synthetized at 30° C. and 200-220 r/min, and fermentation is carried out for 24-72 h.

10. The method of claim 9, wherein the fermentation system contains glucose, glycerol, (NH₄)₂SO₄, K₂HPO₄·3H₂O, KH₂PO₄, MgSO₄·7H₂O, citric acid, vitamin B1, yeast extract, ascorbic acid, and betaine.
